(19) United States
(12) Patent Application Publication
(10) Pub. No.: US 2021/0060573 A1
(43) Pub. Date: Mar. 4, 2021

(54) CONVEYING SCREW FOR FOOD WASTE DISPOSAL DEVICE

(71) Applicant: Su Yun KIM, Gyeonggi-do (KR)

(72) Inventor: Su Yun KIM, Gyeonggi-do (KR)

(21) Appl. No.: 16/645,313

(22) PCT Filed: Sep. 6, 2018
(86) PCT No.: PCT/KR2018/010392

§ 371 Date: Mar. 6, 2020

(30) Foreign Application Priority Data
Sep. 6, 2017 (KR) 20-2017-0004706

(51) Int. Cl. B02C 18/00; B65G 33/30; B65G 33/10; B65G 33/26; B02C 18/14; B02C 23/28; B09B 3/00; A61L 2/07

(52) U.S. Cl. CPC B02C 18/0092; B65G 33/30; B65G 33/10; B65G 33/26; A61L 2/07; B02C 18/145; B02C 23/28; B09B 3/0083; B02C 18/142

(57) ABSTRACT

A conveying screw for a food waste disposal device that can stir, crush and cut food waste generated from homes or restaurants, while transferring the food waste, to reduce the amount of the food waste, includes a rotational shaft, and a plurality of spiral rotor blades installed to an outer peripheral surface of the rotational shaft in a spiral pattern to stir, crush and cut food waste. Each of the rotor blades is provided with a plurality of air supply holes penetrating the rotor blade to supply external air to the food waste between the opposite rotor blades, thereby generating bubbles from the food waste and then discharging vapor.

### BACKGROUND

The present invention relates to a conveying screw, and more particularly, to a conveying screw for a food waste disposal device that can stir, crush and cut food waste generated from homes or restaurants, while transferring the food waste, to reduce the amount of the food waste.

In general, food waste generated from apartment houses or feeding facilities of schools or military camps holds about 30% of the total food waste.

About 5 tons food waste is recently generated in a day, and such a waste generation rate is the primary source of environmental pollution due to population growth and intensified urbanization.

A disposal method of the food waste generally includes a dewatering method, a drying method and a fermenting method.

The dewatering method is to remove water from the food waste by compaction, crushing and high-speed rotation, and is suitable for homes which generates a small quantity of food waste, but is unsuitable for restaurants or feeding facilities which generate a significant quantity of food waste.

The drying and decomposing method is to remove water from the food waste by heating and drying the food waste, which can significantly reduce the volume of the food waste.

In this instance, the internal temperature of a food waste disposal apparatus is maintained at 90° C. after about one hour passed, and 99.9% sterilization through heating can be obtained.

The above drying and decomposing method generally includes a common drying process of heating the inside of a drying tank to a given temperature by hot air or a heater so as to evaporate the moisture contained in the food waste, a drying and crushing process of inputting the food waste into a drying tank and feeding hot vapor onto an outer peripheral surface of the drying tank, while rotating the drying tank, to indirectly heat the food waste at a given temperature, and a fermenting and drying process of mounting a hot blower or a heater inside a fermenting tank to simultaneously perform the fermentation and the drying by use of a temperature higher than a fermenting temperature.

Although not shown in the drawings, a stirring and crushing method using a conveying screw having a rotational shaft provided with tripod-type rotor blades has problems of lowering a stirring efficiency due to a structural feature of the rotor blades which cannot smoothly stir and crush the food waste, and being unable to crush or cut the food waste including shellfish, plant roots and animal bones by the stirring screw.

The stirring screw is installed in a chamber which receives the food waste therein, and the food waste is stuck onto the inner surface of the chamber by the heat generated from the heater to produce the crust of overcooked food.

In the case of producing the crust of overcooked food, carbonization of the food waste causes bad smell. When the heat of the heater is transferred to the food waste, a heat transfer rate is lowered due to a carbonized layer. In addition, the blackened food waste results in production of benzopyrene (carcinogenic to humans—Group 1).

Accordingly, when the food waste is dried or fermented, the bad smell or various toxic gases generated from the inside of the fermenting and drying chamber are discharged to the outside which pollutes the environment.

If subsidiary equipment, such as a deodorization tower or a purification filter, is provided, it significantly increases costs required for purification.

### SUMMARY OF THE INVENTION

Accordingly, the invention has been made in view of the above-mentioned problems occurring in the related art, and one object of the invention is to provide a conveying screw for a food waste disposal device that can stir, crush and cut food waste while the food waste is smoothly feeding along the conveying screw, thereby preventing the food waste from being stuck onto a bottom surface of a chamber due to heat generated from a heater.

Another object of the invention is to provide a conveying screw for a food waste disposal device, in which a plurality of segment rotor blades are integrally installed to the conveying screw, thereby replacing only the rotor blade which is worn out or damaged.

Still another object of the invention is to provide a conveying screw for a food waste disposal device that can feed external air to hot and wet food waste so as to transfer heat to the food waste and form a bubble layer therein, while the food waste is stirred and crushed by a plurality of spiral rotor blades, thereby shortening a processing time and minimizing discharge of bad smell or toxic gases due to condensation of vapor.

To accomplish the above-mentioned object, according to a first aspect of the present invention, there is provided a conveying screw for a food waste disposal device, the conveying screw including: a rotational shaft; and a plurality of spiral rotor blades installed to an outer peripheral surface of the rotational shaft in a spiral pattern to stir, crush and cut food waste, wherein each of the rotor blades is provided with a plurality of air supply holes penetrating the rotor blade to supply external air to the food waste between the opposite rotor blades, thereby generating bubbles from the food waste and then discharging vapor.

The invention has the following advantages.

If the rotor blades are worn out or damaged in the process of transferring the food waste, which is generated from homes or restaurants, by the conveying screw designed to stir, crush and cut the food waste, only the rotor blade of interest can be easily replaced to reduce a maintenance cost.

Also, while the wet food waste is stirred and crushed by the plurality of spiral rotor blades, the external air is supplied to the hot and wet food waste to generate bubbles, and the vapor produced by the bubbles passes through the air-cooled condenser and the water-cooled condenser in order, thereby the environment from being polluted due to the discharge of bad smell or toxic gases and thus providing a user with the reliability.

In addition, as the output of the food waste is minimized (a reduction rate is about 83 to 87%), the conveying screw has a practical value.

### BRIEF DESCRIPTION OF DRAWINGS

FIG. 1 is a perspective view illustrating a conveying screw for a food waste disposal device according to an embodiment of the invention;

FIG. 2 is an exploded perspective view schematically illustrating a food waste disposal device to which the conveying screw in FIG. 1 is applied;

FIG. 3 is a view illustrating an operating state of the conveying screw in FIG. 1;

FIG. 4 is a view illustrating generation of bubbles in food waste by feeding air to the food waste through air through air supply holes formed in rotor blades of the conveying screw in FIG. 1 and discharge of hot vapor generated by heat of a heater; and

FIG. 5 is a cross-sectional view illustrating an operating state of the conveying screw in FIG. 1.

### DETAILED DESCRIPTION OF THE INVENTION

Hereinafter, a conveying screw for a food waste disposal device according to preferred embodiments of the invention will be described in detail with reference to the accompanying drawings.

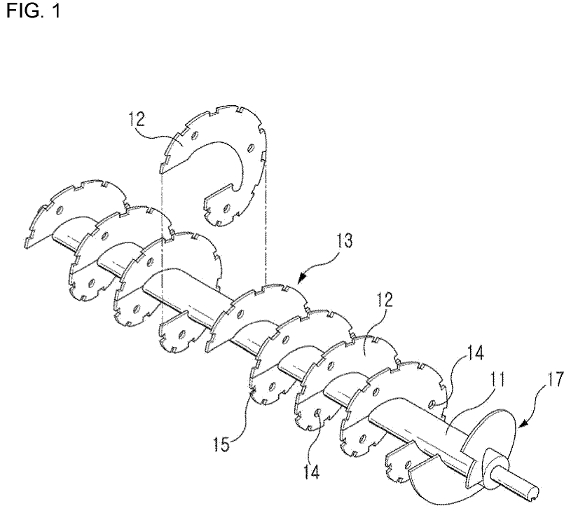

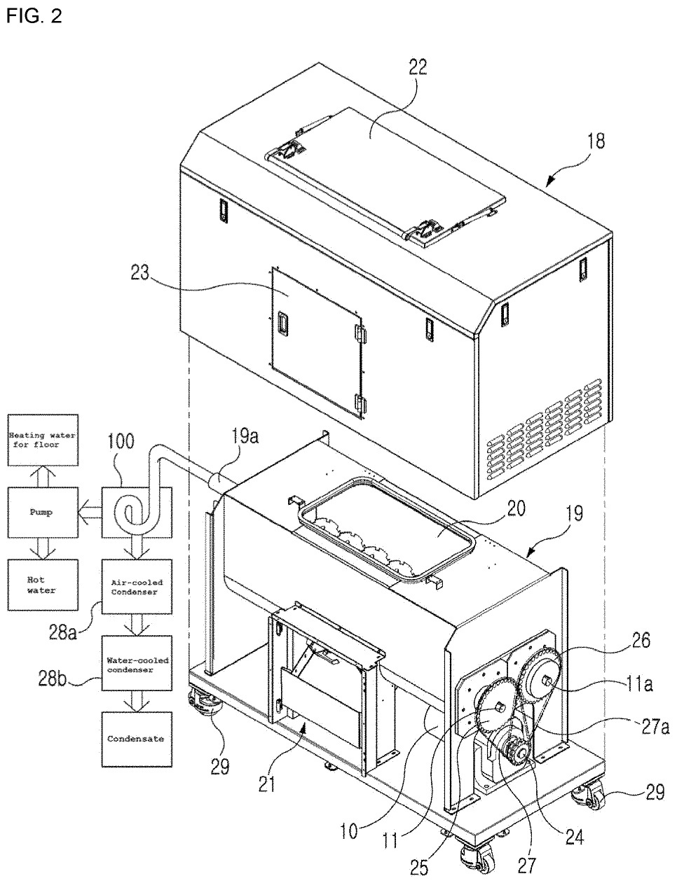

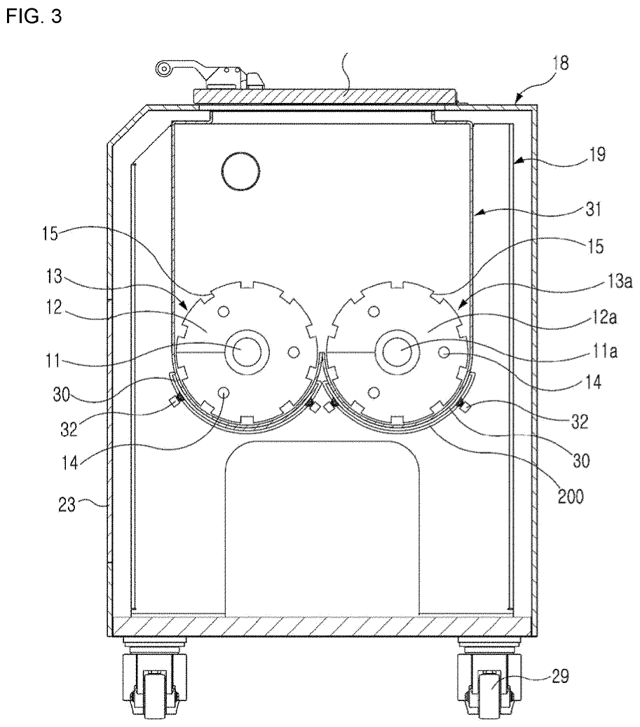

Referring to FIGS. 1 to 5, the conveying screw 13 for the food waste disposal device according to one embodiment of the invention includes a rotational shaft 11 engaged to and rotated by a driving motor 10, and a plurality of spiral rotor blades 12 installed to an outer peripheral surface of the rotational shaft 11 in a spiral pattern to stir, crush and cut food waste containing water which is generated from homes or restaurants, while the food waste is transferring in one way.

Each of the rotor blades is provided with a plurality of air supply holes penetrating the rotor blade to supply external air to the food waste containing the water which is stirring, crushing and cutting by the conveying screw 13, thereby discharging vapor due to generation of bubbles in the food waste.

Preferably, each of the rotor blades 12 having a desired thickness is provided with a plurality of tooth portions 15 formed on an outer edge of the rotor blade at regular intervals to cut the food waste while the food waste is transferring, stirring and crushing.

The rotor blades 12 are formed in a segment type to form a Moebius strip, an inner edge of each rotor blade 12 is closely fixed to an outer peripheral surface of the rotational shaft 11, and both ends of the adjacent rotor blades are joined to each other to form a spiral pattern

In this instance, the rotor blades 12 are fixed to the outer peripheral surface of the rotational shaft 11 by welding or the like, and both ends of the adjacent rotor blades 12 are joined to each other. This structure is widely known in the art, and thus the detailed description thereof will be omitted herein.

In the case of converting a transfer direction of the food waste to be transferred by rotation of the conveying screw 13, the conveying screw 13 may further include a direction converting plate 17 provided at one end of the rotational shaft 11 to transfer the food waste to an adjacent conveying screw 13a.

Specifically, when the food waste reaches the end of the conveying screw 13, the transfer direction of the food waste can be easily converted by the direction converting plate 17, without staying in the end of the shaft, so that the food waste is smoothly transferred to the adjacent conveying screw 13a.

A reference numeral 18 denotes a casing having a lid 22 and a door 23 to open or close an inlet port 20 and an outlet port 21 of a housing 19, 24 denotes a two-step driving gear coupled to a shaft of the driving motor 10, 25 denotes a first driven gear engaged to the rotational shaft 11 of the conveying screw 13, 26 denotes a second driven gear engaged to a rotational shaft 11a of the conveying screw 13a, 27 and 27a denote endless chains for connecting the driving gear 24 and the first and second driven gears 25 and 26, respectively, to rotate the conveying screws 13 and 13a by rotation of the driving motor 10, 28a and 28b denote condensers of air-cooled and water-cooled types, respectively, 29 denotes casters mounted to a bottom surface of the housing, 30 denotes an electric heater for drying the food waste to be transferred by the conveying screws 13 and 13a, and 31 denotes a chamber for forming a transfer passage of the food waste to be transferred by the conveying screws 13 and 13a.

The operation of the conveying screw for the food waste disposal device according to the embodiment of the invention will now be described with reference to the accompanying drawings.

As illustrated in FIGS. 1 to 5, when a power switch (not illustrated) is turned by a user to activate the driving motor 10, the two-step driving gear 24 engaged to the shaft of the driving motor 10 is rotated, and the first and second driven gears 25 and 26 connected to the driving gear 24 by the endless chains 27 and 27a are rotated.

In this instance, since the rotational shafts 11 and 11a of the conveying screws 13 and 13a are rotated at a reduction ratio of 1:60 with respect to the rotational speed of the driving motor 10, the spiral conveying screws 13 and 13a are rotated at a low speed.

As described above, as the driving motor 10 is actuated and then the first and second driven gears 25 and 26 are rotated by rotation of the two-step driving gear 24, the spiral conveying screws 13 and 13a are rotated in an opposite direction. To this end, the spiral conveying screws 13 and 13a are disposed in an opposite direction.

Accordingly, when a proper amount of the food waste containing water (a moisture content is 82 to 85%) which is generated from homes or restaurants is put in the inlet port 20 of the housing 19 in a state in which the lid 22 mounted to a top surface of the casing 18 is opened, the food waste is dropped between the rotator blades 12 and 12a of the pair of conveying screws 13 and 13a.

The food waste to be stirred, crushed and cut by the conveying screws 13 and 13a may include shellfish, plant roots and animal bones by the stirring screw.

Specifically, the food waste dropped between the rotor blades 12 and 12a is stirred and crushed while being transferred in one way by the pair of rotor blades 12 and 12a.

In this instance, since the conveying screw has a cutting function, the food waste can be further crushed and cut by the plurality of tooth portions 15 which are formed on the outer edge of the rotor blade at regular intervals.

The food waste to be stirred and crushed by the rotation of the pair of rotor blades 12 and 12a is horizontally transferred along the conveying screws 13 and 13a in the chamber 31.

Since the rotor blades 12 and 12a are segmented and fixed to the rotational shafts 11 and 11a in the spiral pattern, when the rotor blades 12 and 12a for stirring, crushing and cutting the food waste may be worn out or damaged, only the rotor blade of interest is replaced to reduce a maintenance cost.

The food waste supplied into the chamber 31 is continuously transferred, stirred and crushed by the spiral rotor blades 12 and 12a of the conveying screws 13 and 13a, and simultaneously, the food waste is dried by the heat of the heater 30, thereby preventing the food waste from staying and being stuck to the bottom surface of the chamber 31.

As described above, the moisture generated from the food waste which is transferred by the spiral conveying screws 13 and 13a and is stirred and crushed by the rotor blades 12 and 12a can be dried by the heat of the heater 30 which is mounted on the bottom surface of the chamber 31.

Specifically, if the power is applied to the heater 30 via a power supply terminal 32 provided on a bottom surface of the heater 30, the heat of a given temperature generated from a planar heating member built in the heater 30 is transferred to the bottom surface of the chamber 31.

Since the chamber 31 is heated by the heater 30, the heat is transferred to the rotor blades 12 and 12a of the spiral conveying screws 13 and 13a which are spaced apart from the chamber 31. Therefore, the inside of the housing 19 is under a dry atmosphere, and the food waste to be crushed and transferred in a horizontal direction in the chamber 31 can be dried.

In this instance, since the heater 30 is formed in a modular type and is detachably mounted to the bottom surface of the chamber 31, if the heater 30 is worn out or damaged, only the heater of interest can be replaced, thereby minimizing a maintenance cost.

A pocket 200 including the heater is mounted on the bottom surface of the chamber 31, and is sealed to maintain the inside under a vacuum atmosphere, thereby preventing a heat loss when the heat is transferred from the planar heating member to the food waste and minimizing the heater 30 from being damaged. Therefore, the efficiency of the thermal energy can be maximized by the pocket.

Specifically, the water of the food waste or debris of the food waste is prevented from coming into the heater 30 by the pocket 200 to prevent the heater 30 from being oxidized and corroded and thus remarkably extend a lifetime of the heater 30.

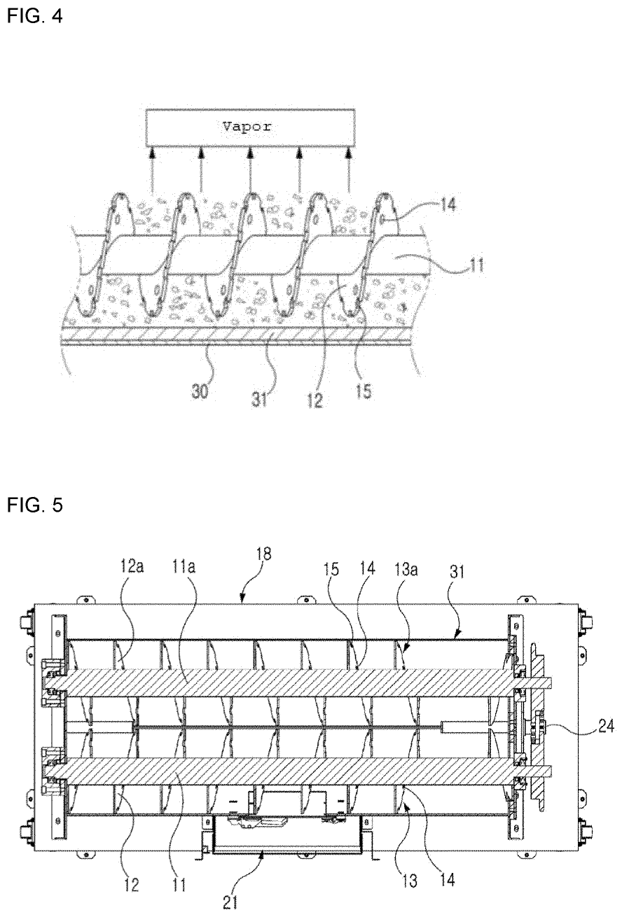

As illustrated in FIG. 4, in the case where the food waste is dried by the heater 30 while being transferred, stirred and crushed by rotation of the spiral conveying screws 13 and 13a, the external air fed to the housing 18 by an air pump (not illustrated) can be supplied to the food waste to be stirred and crushed between the opposite rotor blades 12 and 12a via the air supply holes 14 which are formed in the rotor blades 12 and 12a.

The external air is supplied to the food waste containing the water between the opposite rotor blades 12 and 12a via the air supply holes 14 of the rotor blades 12, and the water is evaporated by the heat contained in the hot and wet food waste in the chamber 31 in cooperation with the heat of the heater 30.

Specifically, bubbles are produced in the food waste by the high temperature (about 190° C.) contained in the food waste and the high moisture (a moisture ratio is maintained at about 82%) to evaporate the hot moisture contained in the food waste.

The vapor generated from the food waste is discharged outwardly from a vapor discharge pipe 19a provided on an upper portion of the housing 19.

The hot vapor (about 190° C.) discharged from the housing 19 passes through a water tank 100 connected to the vapor discharge pipe 19a.

Specifically, if the vapor of high temperature and humidity discharged from the housing 19 passes through the water tank 100 via the vapor discharge pipe 19a, the water of the water tank 100 is converted to hot water by heat exchange of the water in the water tank with the hot vapor.

In this instance, the hot water of the water tank 100 can be supplied to a location (restaurants or stables) requiring for hot water or heating water for a floor by a pump, thereby reducing hot water generating costs or heating costs which are financial burden.

As the moisture of the food water passing through the water tank 100 contains bad smell, the moisture is primarily condensed by the air-cooled condenser 28a, thereby removing the bad smell contained in the moisture.

The moisture which is primarily condensed by the air-cooled condenser 28a passes through the water-cooled condenser 28b, to remove a great part of the bad smell (about 90 to 95%) contained in the moisture.

Finally, the condensate produced by the moisture passing through the water-cooled condenser 28b is discharged to a drain.

The housing 19 enclosed by the casing 18 having the lid 22 and the door 23 is sealed, and only the hot vapor generated from the food waste passes the water tank 100 via the vapor discharge pipe 19a, and then is condensed and purified by the air-cooled and water-cooled condensers 28a and 28b.

Therefore, a great part of the bad smell or toxic gases generated in the process of stirring, crushing, transferring and drying the food waste containing the moisture is condensed and purified in the housing, and the condensate generated from the condensers 28a and 28b is discharged to the drain, thereby minimizing the bad smell produced from the food waste from being discharged to the outside.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

The food waste is stirred, crushed and cut by the conveying screw, while the food waste is smoothly feeding along the conveying screw, thereby preventing the food waste from being stuck onto the bottom surface of the chamber due to the heat generated from the heater. Also, since the plurality of segment rotor blades are integrally installed to the conveying screw, only the worn or damaged rotor blade can be easily replaced. In addition, the external air is supplied to the hot and wet food waste so as to transfer the heat to the food waste and form the bubble layer therein, while the food waste is stirred and crushed by the plurality of spiral rotor blades, thereby shortening the processing time. The vapor to be discharged is condensed to minimize the discharge of the bad smell or toxic gases.

1. A conveying screw for a food waste disposal device, the conveying screw comprising:
a rotational shaft; and
a plurality of spiral rotor blades installed to an outer peripheral surface of the rotational shaft in a spiral pattern to stir, crush and cut food waste,
wherein each of the rotor blades is provided with a plurality of air supply holes penetrating the rotor blade to supply external air to the food waste between the opposite rotor blades, thereby generating bubbles from the food waste and then discharging vapor.

2. The conveying screw for the food waste disposal device according to claim 1, wherein each of the rotor blades is provided with a plurality of tooth portions formed on an outer edge of the rotor blade at regular intervals to cut the food waste.

3. The conveying screw for the food waste disposal device according to claim 1, wherein the rotor blades are formed in a segment type to form a Moebius strip, an inner edge of each rotor blade is closely fixed to an outer peripheral surface of the rotational shaft, and both ends of the adjacent rotor blades are joined to each other to form a spiral pattern.

4. The conveying screw for the food waste disposal device according to claim 1, further comprising a direction converting plate which is provided at one end of the rotational shaft to transfer the food waste to an adjacent conveying screw, so that a transfer direction of the food waste to be transferred by rotation of the conveying screw is converted.

5. The conveying screw for the food waste disposal device according to claim 2, wherein the rotor blades are formed in a segment type to form a Moebius strip, an inner edge of each rotor blade is closely fixed to an outer peripheral surface of the rotational shaft, and both ends of the adjacent rotor blades are joined to each other to form a spiral pattern.

6. The conveying screw for the food waste disposal device according to claim 2, further comprising a direction converting plate which is provided at one end of the rotational shaft to transfer the food waste to an adjacent conveying screw, so that a transfer direction of the food waste to be transferred by rotation of the conveying screw is converted.
